(19) United States
(12) Patent Application Publication
(10) Pub. No.: US 2008/0249592 A1
(43) Pub. Date: Oct. 9, 2008

(54) Resiliently conformable applicator structure

(76) Inventor: Robert G. James, Bakersfield, CA (US)

Correspondence Address: I. Michael Bak-Boychuk;Attorney at Law, P.O. Box 32501, Long Beach, CA, 90832, US

(21) Appl. No.: 11/784,252

(22) Filed: Apr. 6, 2007

(51) Int. Cl. A61N 2/08

(52) U.S. Cl. 607/100

(57) ABSTRACT

A fluid filled applicator conformed to position a source of electromagnetic radiation adjacent a portion of human anatomy to interpose a fluid of known characteristics between the source and the adjacent tissue includes in its first form a generally resilient envelope rendered conformable to the body surface to which it is applied by adjusting the internal pressure therein. In an alternative form the fluid is confined to a selected depth of a vertical container and the limb or body part is then immersed therein while contained in a flexible sheath. The source may then be applied to any selected exterior portion of the container.

### BACKGROUND OF THE INVENTION

1. Field of the Invention

The present invention relates to resiliently conformable devices and more particularly to resilient charge applicators pressurized for conforming fit with selected surfaces and portions of the human anatomy.

2. Description of the Prior Art

Devices that in one manner or another selectively direct an electromagnetic field or charge to parts of human anatomy have been known in the past, including devices that apply pulsed signals useful in stimulating bone tissue repair like those described in U.S. Pat. Nos. 5,217,009 to Kronberg; and 4,430,999 to Brighton, et al.; devices for stimulating muscle by electrical charge or current like that exemplified in U.S. Pat. No. 4,249,537 to Lee et al.; and devices that apply beneficial light like that described in U.S. Pat. Nos. 5,913,883 to Alexander et al., 6,069,066 to Chen et al., 6,471,716 to Pecukonis; or those described in U.S. Pat. Nos. 6,378,760 and 7,130, 689 issued earlier to this inventor. In each instance these fields, currents or other electromagnetic charges are delivered to the treated body site by a hand-held or mechanically mounted device brought generally proximate the patient's limb or area of interest. As result of this general placement imprecise and uneven distance distributions are inherent between the face of the emitting source and the receiving body surface portions which, because of the exponential field strength effect with distance, often affect the efficacy of treating process.

To overcome these geometric imprecisions some devices in the above described prior art like the '883 and '066 patents, or devices like those described in U.S. Pat. No. 6,443,978 issued to Zharov, distribute an array of emitting sources over convolved surfaces that generally conform, or are conformable, to the anatomy that is to be treated. While suitable for the purposes intended, these prior art structures either entail plastically conformable surfaces, or an array of surface shapes adapted for various surface matching, that are either numerically burdensome or require manipulative strength and dexterity to effect the desired matching panel conformation.

Moreover, many charge devices, including those described in my previous '760 and '689 patents, interpose a controlled volume of liquid or gas between the charge source and the body surface, a configuration that may be masked or affected by the air gaps therebetween that variably attenuate any electromagnetic transmission into the treated tissue. This variability adversely affect the treating efficacy of the device while also encumbering the treating staff with the repeating need for device re-positioning as the patient's posture changes in the course of any treatment regimen. A mechanism that conveniently fixes the charge source and its associated intervening liquid or gas volumes relative the treated body portion is therefore extensively desired and it is one such mechanism that is described herein.

### SUMMARY OF THE INVENTION

Accordingly, it is the general purpose and object of the present invention to provide a resiliently deformable applicator enclosure conformed for selected internal pressurization.

Other objects of the invention are to provide a resilient envelope dimensioned to surround a selected portion of the anatomy of a person.

Further objects of the invention are to provide a closed liquid volume between the applicator electrodes of a electrical stimulation device and the treated portion of a person.

Yet additional objects of the invention are to provide a compliant fluid containment envelope provided with an electrode on one surface thereof and internally pressurized for conformable contact with selected portions of a human anatomy.

Briefly, these and other objects are accomplished within the first embodiment of the present invention by providing a first resilient pouch or envelope defined by a first and a second membrane each of a generally rectangular planform joined at their respective peripheries to the other to form an enclosure or envelope confining a volume fluid therebetween. Preferably the fluid pressure within the enclosure is maintained at a level at which compliant contact can be effected between one of the membranes thereof and a selected portion of human anatomy so that any field or radiation pattern from an electrode on the exterior of the other opposed membrane encounters few or no air gaps in its propagation path to the body surface. In this manner the interposing matter between the electrode and the tissue that is treated comprises primarily the two membranes themselves and the fluid confined therebetween, thus assuring the conductivity and any electropotential effects in the transmission to the treated site. To assure this envelope compliance a fluid transfer path may be provided across a pressure relieving valve into the interior of a collapsed second storage envelope, the relief valve further including a manually operable mechanism to allow the fluid stored therein to drain back, by gravity, into the first envelope. By selecting the vertical drop between the first and second envelopes in the course of this draining process the internal pressure bias and the resulting compliance of the first envelope are conveniently adjusted.

In a second embodiment the internal pressure of the fluid within the enclosure may be controlled by a manually adjustable accumulator structure in which the accumulator pressure is adjusted by pneumatically increasing the pressure on the exterior of the accumulator surface. In this manner the internal pressure of the fluid in the envelope may be controlled to meet the compliance needs of the body surface that is treated.

A third embodiment of the invention utilizes an open container having the electrode or emitting source adhered to the exterior of one wall thereof with the limb that is to be treated then inserted into a closely compliant sheath and thus covered then immersed in the fluid in the container. The pressure head obtained by the depth of the limb immersion then assures close contact between the sheath and the limb thereby limiting any air gaps between the source and the area treated.

Those skilled in the art will appreciate that each of the foregoing embodiments limits the incidence of unwanted gaps along the transmission path of a charge or current through human tissue. In this manner only the normal variations in the insulative or dielectric properties of human skin provide the operational variables and these can be controlled by hydrating ointments or other creams that either promote or reduce conductivity. In consequence the charge potential and also the current levels and densities can be accurately determined, an advantage particularly useful in the functional modalities taught in this inventor's prior U.S. Pat. Nos. 6,378,760 and 7,130,689. Thus while broadly useful the above summarized inventive embodiments are particularly advantageous in the processes described in the '760 and '689 patents and similarly operating processes.

### BRIEF DESCRIPTION OF THE DRAWINGS

FIG. 1 is a perspective illustration of the first embodiment of the inventive applicator structure in place on a limb of a person;

FIG. 2 is a diagrammatic illustration of the structure useful with the first embodiment of the inventive applicator structure;

FIG. 3 is a sectional detail of the inventive relief valve shown in FIGS. 1 and 2;

FIG. 4 is a further diagrammatic illustration of the first embodiment of the inventive applicator structure having portions thereof positioned at various selected relative elevations for controlling the internal fluid pressure therein;

FIG. 5 is yet another perspective illustration including portions in partial section, illustrating a second embodiment of the inventive applicator structure;

FIG. 6 is a sectional side view taken along line 6-6 of FIG. 5, illustrating an inventive accumulator useful herein;

FIG. 7 is a further perspective illustration, including portions in partial section, illustrating a third embodiment of the inventive applicator structure; and

FIG. 8 is a further sectional side view detail taken along line 8-8 of FIG. 7.

### DESCRIPTION OF THE PREFERRED EMBODIMENTS

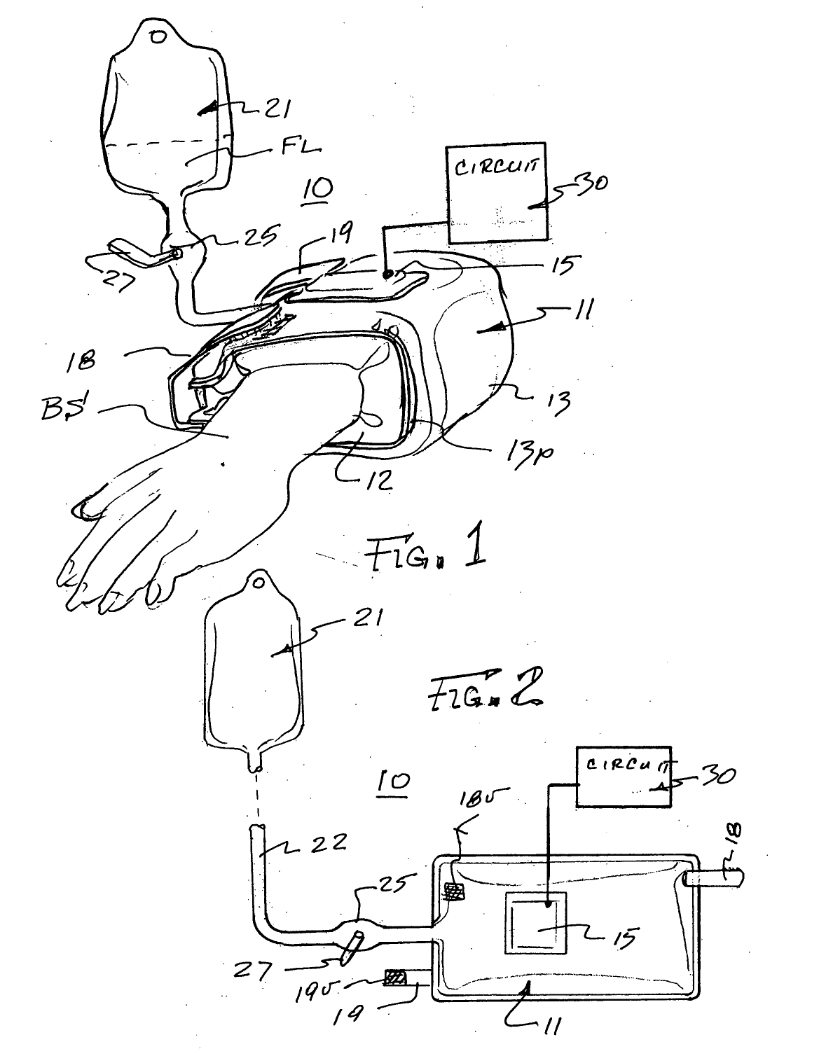

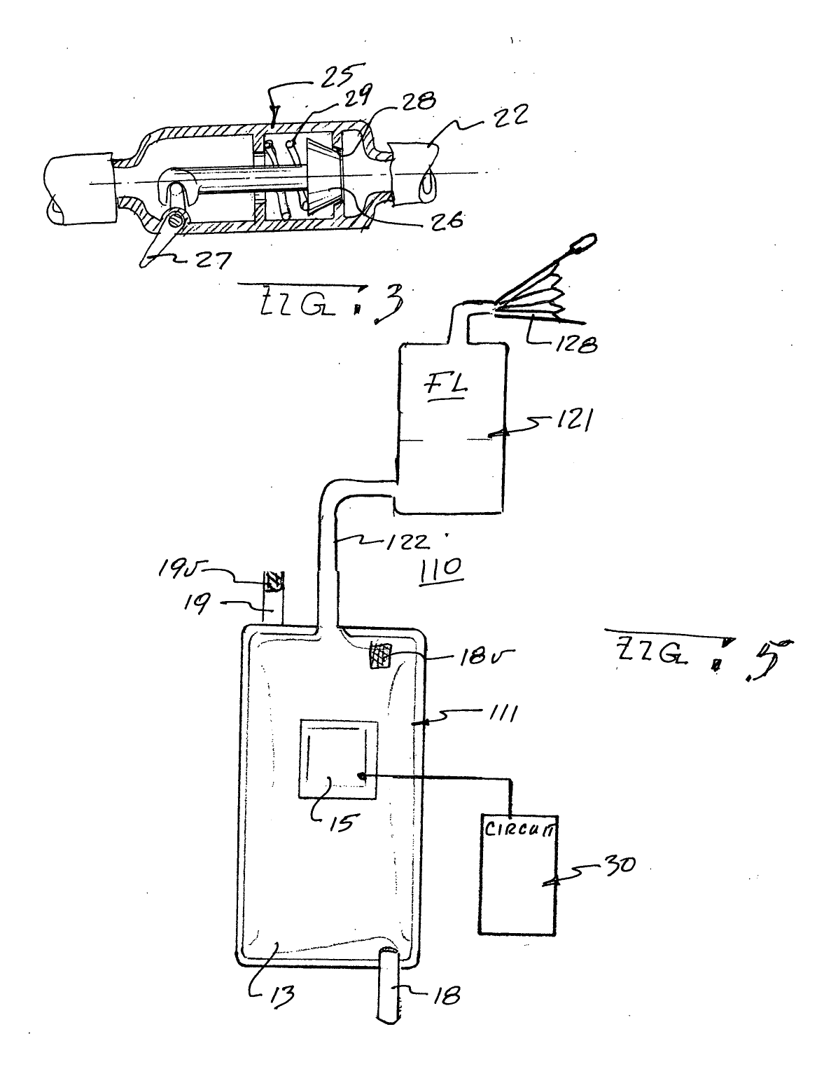

As shown in FIGS. 1-4 the inventive applicator structure, in accordance with its first embodiment generally designated by the numeral 10, comprises an applicator envelope 11 defined by a resilient upper membrane 12 and a substantially equally dimensioned lower resilient membrane 13 joined to each other at their respective peripheries 12p and 13p to form a fluid impervious enclosure. Preferably membranes 12 and 13 are each of a rectangular planform having the longer dimension thereof equal or greater than the larger peripheral arcs of the major areas of human anatomy such that the weight of any fluid in the envelope will result in a conforming contact between the lower membrane 13 and the adjacent body surface BS. By eliminating most air gaps this conforming contact then assures a repeatable transmission path between a planar electrode 15 adhered to the exterior of the lower membrane 13 and the subjacent body surface, a path that includes the upper membrane, the height of the fluid FL confined in the envelope 11 and the lower membrane 13, but very little else.

To further assure conforming compliance of envelope 11 with the body surface BS on which it rests the first embodiment 10 is provided with a gravity assisted pressurization arrangement including a closed, resilient overflow pouch 21 in which an excess of the fluid FL is stored. A flexible fluid transfer hose 22 provided with a manually releasable check valve mechanism 25 connects pouch 21 to envelope 11 to allow the deployment of the pouch at various elevations relative the envelope and thereby modify the internal fluid pressure in the envelope. This transfer may be effected by providing an enlarged handle 27 on a valve pindle 26 opposed by the relief spring 29. Handle 27 can then be manipulated through the resilient walls of the hose 22 to lift the pindle 26 from its seat 28 while the pouch 21 is raised or lowered to heights I, II or III to raise or lower the internal fluid pressure within envelope 11. Once the desired pressure level is obtained the valve handle 27 is released, re-seating pindle 26 on its seat 28 which then fixes the interior pressure in envelope 11.

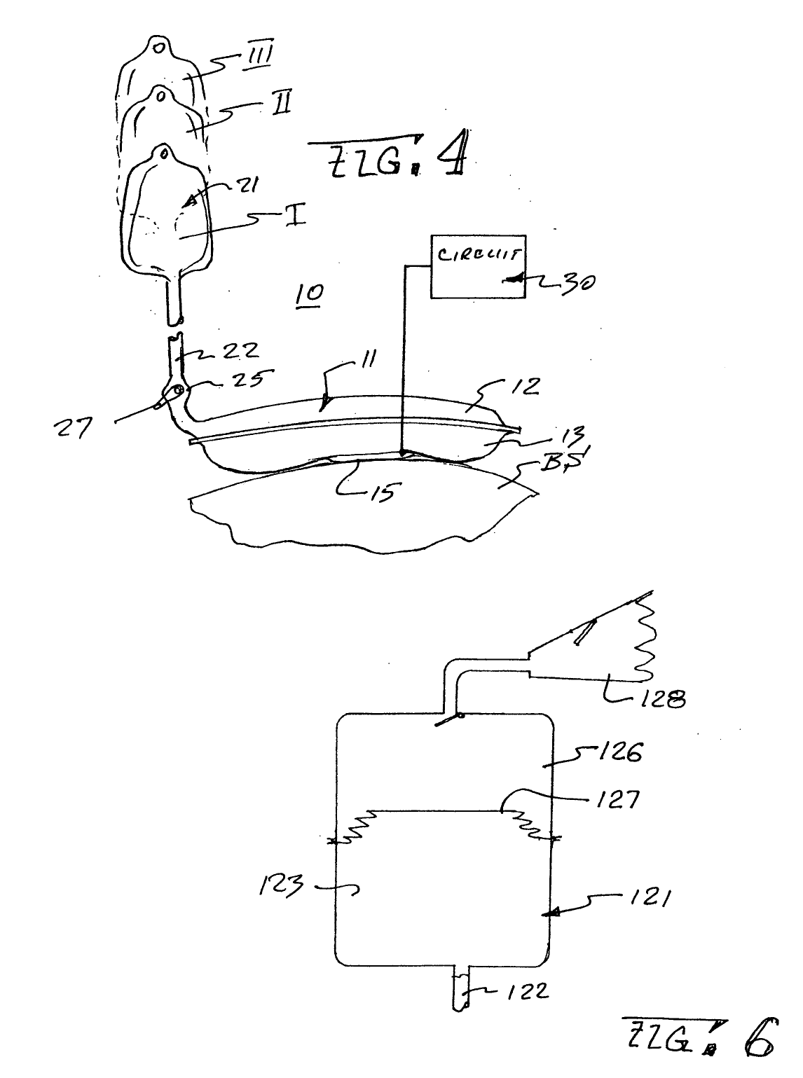

Those in the art will appreciate that the foregoing arrangement fully and completely confines the whole of the fluid FL within the common interiors of envelope 11, hose 22 and the pouch 21. In this manner the chemical constituents of the fluid FL remain uncontaminated, allowing for the accurate transmission therethrough of the charges, fields or currents issued from circuit 30 like that earlier described by this inventor in the U.S. Pat. Nos. 6,378,760 and 7,130,689. Moreover, for those instances where the gaseous nature of the fluid FL precludes effective pressure, and therefore compliance, control by gravity gradient the instant teachings include a second embodiment, shown in FIGS. 5 and 6 and generally designated by the numeral 110, in which a resilient envelope 111 is connected by a hose 122 to an accumulator 121. Accumulator 121, in turn, includes a pressure biasing chamber 126 separated by a flexible wall 127 from the common interior 123 communicating into envelope 111, the biasing chamber 126 further including a manually operable pump 128 by which its internal pressure can be raised. This added pressure in the biasing chamber 126, together with the elastic tension in wall 127, then determine the pressure in the envelope.

Like numbered parts functioning in a like manner to that previously described, the foregoing arrangement insures a compliant contact between the lower membrane 13 of envelope 111 and the adjacent body surface. Moreover, the fluid constituents confined within envelope 111 between membranes 12 and 13, and therefore the medium through which any emission-absorption cascades may occur, remain uncontaminated thereby insuring the efficacy of the process. As before, this cascade or other charge originates at electrode 15 radiating along a path that includes the fluid FL with its desired constituents and then into the tissue.

It will be appreciated that both the foregoing embodiments conveniently conform to various body shapes by simply adjusting the internal fluid pressure in either envelope 11 or 111. Then, once the desired surface contact is achieved either the handle 27 on the relief valve is released, reseating the valve, or the desired biasing pressure is achieved in cavity 126 by manipulating pump 128. This conforming alignment can then be retained by a set of straps 18 and 19 extending from the peripheral edge 13p, each provided with selectively engageable hook and pile surfaces 18v and 19v like those sold under the mark or style VELCRO. In this manner long treatment periods can be comfortably sustained.

Those skilled in the art will further appreciated that other treatment regimens either using light or laser sources or even a heat source are easily accommodated in these same structures. For example, in those instances where the treatment entails small current or heat flows into the treated tissue delivered from electrode or source 15 then its positioning is best effected on the exterior of the lower membrane 13. Of course, the designation of ‘lower’ and ‘upper’ when referring to the membranes here is for descriptive purposes only as the envelopes 11 and 111 and their securement may be rendered fully reversible, the intent being that both direct skin contact with the electrode or a mediated path that includes propagation through fluid FL are easily accommodated in the same structure.

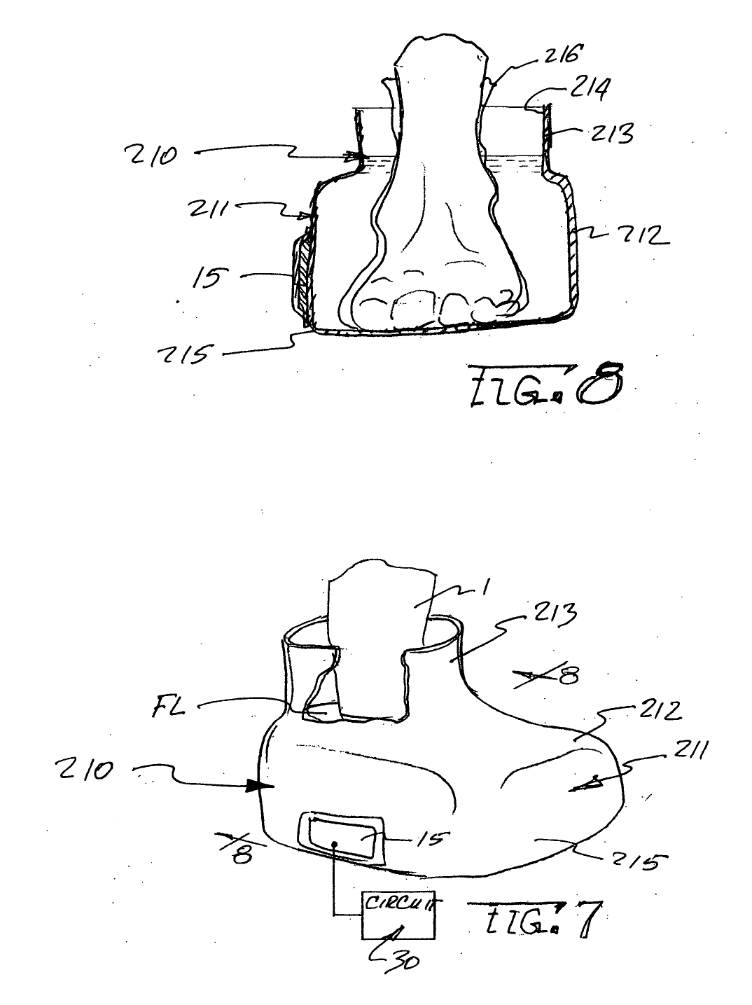

By reference to FIGS. 7 and 8 a third embodiment of the present invention, generally designated by the numeral 210, is conformed generally as a container in which an extremity can be immersed. This fluid container appears as a hollow, generally rigid, enclosure 211 defined by a lower chamber 212 communicating into a vertical, somewhat narrower, stack 213, chamber 212 and stack 213 then in combination defining a volume useful to store fluid FL into which a limb LB of a patient can be extended through an upper opening 214 to be immersed. Of course, the height of the fluid FL and its density will then define the pressure gradient around the immersed limb either to determine the local fluid pressure on the patient's skin or to provide sufficient external pressure on the surfaces of an impervious flexible bag 216 covering the limb so that all the folds thereof are fully collapsed to drive any trapped air therefrom. Once again, the electrode 15 may be then affixed at any desired exterior surface portion of the lower exterior surfaces 215 of chamber 212, thereby defining a repeatable transmission configuration between the electrode and the treated part of the person.

Those skilled in the art will appreciate that each of the foregoing embodiments seeks to minimize the volumes trapped within the spurious foldings of a membrane as it conforms with the various anatomical topographies of the patient by selecting the desired pressure differential thereacross. These benefits are particularly significant in a setting where the patient's skin, itself, has lost its local integrity by injury, disease or condition and a high skin impedance is necessary to insure effective treatment. Where higher current densities are a preferred part of the regimen direct immersion is then obtained. In this manner a widely useful application arrangement is obtained that is useful with a wide range of regimens.

Obviously many modifications and variations of the instant invention can be effected without departing from the spirit of the teachings herein. It is therefore intended that the scope of the invention be determined solely by the claims appended hereto.

It is claimed:

1. A resilient applicator assembly useful to position a desired volume of fluid adjacent an electromagnetic source and a selected body portion of a person, comprising:
a first resilient envelope conformed to imperviously contain said fluid defined by a first resilient membrane joined at the periphery thereof to the periphery of a second opposingly aligned resilient membrane;
pressurization means connected to said envelope and conformed for manual adjustment of the pressure of said fluid in said envelope; and
attachment means formed on the exterior surface of said first membrane for affixing said source thereto.

2. An applicator assembly according to claim 1, wherein:
said first and second membranes defining said first envelope are each of an elongate planform to cooperatively form therebetween an elongate enclosure, said elongate enclosure including connecting elements at the ends thereof for effecting a surrounding engagement to said person adjacent said selected body portion.

3. An applicator according to claim 1 wherein:
said pressurization means includes a second resilient envelope containing a further portion of said volume of fluid and a selectively enabled fluid conveyance between the first and second envelopes for retaining the internal fluid pressure in said first envelope in accordance with the elevation of said second envelope relative thereto.

4. An applicator according to claim 3, wherein:
said fluid conveyance includes a relief valve conformed for selective manual opening.

5. An applicator assembly according to claim 4, wherein:
said first and second membranes defining said first envelope are each of an elongate planform to cooperatively form therebetween an elongate enclosure, said elongate enclosure including connecting elements at the ends thereof for effecting a surrounding engagement to said person adjacent said selected body portion.

6. An applicator according to claim 5 wherein:
said pressurization means includes an accumulator connected to communicate with said first envelope for containing a further portion of said volume of fluid, said accumulator further including a separated pressure biasing chamber conformed for manual adjustment of the pressure therein.

7. An applicator assembly according to claim 7, wherein:
said pressure biasing chamber includes a manually operable air pump.

8. An applicator assembly according to claim 7, wherein:
said first and second membranes defining said first envelope are each of an elongate planform to cooperatively form therebetween an elongate enclosure, said elongate enclosure including connecting elements at the ends thereof for effecting a surrounding engagement to said person adjacent said selected body portion.

9. An applicator assembly useful for immersing a body portion of a person in a volume of fluid exposed to a source of electromagnetic emissions, comprising:
a hollow lower chamber joined to communicate into the interior of a vertically aligned upper chamber, said lower and upper chambers cooperatively forming a common hollow interior provided with an opening at the upper end thereof for storing said volume of fluid in said interior for receiving through said opening and immersing in said volume of fluid a selected body portion of said person therein; and
attachment means formed on the exterior of said lower hollow chamber for securing said source or electromagnetic emissions thereto.

10. An applicator according to claim 9, further comprising:
a fluid impervious resilient sheath conformed to surround said selected body portion upon the immersion thereof in said volume of fluid.

11. An applicator according to claim 10, wherein:
said sheath is brought into intimate contact with the exterior surfaces of said selected body portion in the course of immersion thereof in said volume of fluid.
